(19) United States
(12) Patent Application Publication
(10) Pub. No.: US 2007/0276387 A1
(43) Pub. Date: Nov. 29, 2007

(54) Implant for fixing two bone parts to be connected to each other

(75) Inventors: Pedro Morales, Tuttlingen (DE); Dieter Weisshaupt, Immendingen (DE); Manfred Dworschak, Duerbheim (DE); Theodor Lutze, Balgheim (DE)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/787,200

(22) Filed: Apr. 12, 2007

(30) Foreign Application Priority Data
Apr. 28, 2006 (DE) 10 2006 021 024.7

(51) Int. Cl. A61B 17/84

(52) U.S. Cl. 606/72

(57) ABSTRACT

An implant for fixing two bone parts to be connected to each other is provided. The implant includes a first plate-shaped engaging element for engaging inner surfaces of the bone parts, at least two tensioning elements fixed on the engaging element and projecting transversely from it, and a second plate-shaped engaging element for engaging outer surfaces of the bone parts. The second engaging element can be tensioned with respect to the first engaging element by means of the tensioning elements brought through the interspace between the bone parts. To improve the ease of handling of the implant during tensioning, it is proposed that the tensioning elements are connected to each other at their end opposite from the first engaging element by a bridge.

This application claims the benefit of German patent application no. 10 2006 021 024.7 filed on Apr. 28, 2006, which is incorporated herein and made a part hereof by reference.

### BACKGROUND OF THE INVENTION

The invention relates to an implant for fixing two bone parts to be connected to each other, with a first plate-shaped engaging element for engaging inner surfaces of the bone parts, with at least two tensioning elements, fixed on said engaging element and projecting transversely from it, and with a second plate-shaped engaging element for engaging outer surfaces of the bone parts, which can be tensioned with respect to the first engaging element by means of the tensioning elements brought through the interspace between the bone parts.

Such an implant is described for example in DE 103 26 690 B4. The engaging elements are in this case of a rectangular form and are tensioned with respect to each other by two tensioning elements in the form of pins, which project through the interspace between the bone parts to be tensioned together. This ensures that the rectangular engaging elements are disposed parallel to this interspace, which is in the form of a gap, between the bone parts and also parallel to each other, that is to say the relative orientation of the engaging elements can be ensured by the provision of two tensioning elements of this kind.

However, with two tensioning elements of this kind on which the second engaging element is displaceably mounted, it is difficult to achieve bracing of the second engaging element in relation to the first engaging element without tilting. In the case of the known device, for this purpose a tensioning tool has to be applied alternately to the two tensioning elements one after the other, in order to displace the second engaging element by a specific distance in the direction of the first engaging element on each of the two tensioning elements. This is laborious, and there is also the risk of instruments, tissue etc. being caught on the free ends of the free tensioning elements, which are of pin form, or the risk of injury being caused by these free ends.

It is an object of the invention to develop an implant of the generic type in such a way that handling is made easier, in particular when the engaging elements are being tensioned.

### SUMMARY OF THE INVENTION

This object is achieved according to the invention in the case of an implant of the type described at the beginning by the tensioning elements being connected to each other at their end opposite from the first engaging element by a bridge.

As a result, the tensioning elements are stabilized and the free ends of the tensioning elements are covered, so as to rule out the possibility of injury occurring at these free ends or something being caught on them.

In particular, it may be provided that the bridge is connected to the tensioning elements so as to be non-displaceable in the tensioning direction. Then the bridge can serve as an abutment for a tensioning tool, by which the second engaging element is tensioned with respect to the first engaging element.

In particular, the tensioning elements may be formed as pins.

For fixing the bridge on the tensioning elements, it is provided according to a preferred embodiment of the invention that the bridge is connected to the tensioning elements in a non-displaceable manner by crimping.

The tensioning elements may be inserted in bores in the bridge, so that an exact parallel guidance of the tensioning elements can be achieved as a result.

In the case of a preferred embodiment of the invention, it is provided that the bridge has an engaging surface for a tensioning device which, with two tensioning tools, can be applied on the one hand to the engaging surface and on the other hand to the outer surface of the second engaging element and tensions the second engaging element with respect to the first engaging element when the tensioning tools are pushed apart.

In this case, it is advantageous in particular if the engaging surface and the tensioning tool engaging it are shaped in such a way that the tensioning tool is supported on the bridge midway between two tensioning elements. In this way a clear supporting point for the tensioning tools is obtained, lying on the bridge midway between the two tensioning elements, and the tensioning forces can therefore act on the second engaging element symmetrically in relation to the tensioning elements.

Furthermore, it is advantageous if the tensioning tool that can be applied to the second engaging element can be applied at two points on the second engaging element which are disposed in the plane defined by two tensioning elements at a distance from each other and away from the midpoint between the two tensioning elements. Altogether, three-point engaging contact is obtained in this way for the tensioning tools, to be specific on the bridge midway between the tensioning elements and on the second engaging element in the plane of the two tensioning elements, but at a distance from the midpoint. This avoids tilting of the second engaging element during the displacement, and the second engaging element can be displaced into the tensioning position without any difficulty.

In particular, the engaging surface on the bridge may extend in an arcuate form, so that the tensioning tool engaging the engaging surface during tensioning is inevitably pushed to the deepest point of the engaging surface, and consequently midway between the two tensioning elements, in particular if the engaging tensioning tool is in a similar way given an arcuate form.

According to a further advantageous configuration, it is provided that the engaging elements carry fixing projections protruding from them and respectively pointing toward the other engaging element and that these fixing projections are inclined in such a way that their distance from the fixing projections on the opposite side of the engaging element increases from the engaging element toward the free end of the fixing projections. Such a configuration has the effect when the two engaging elements are tensioned together that the bone parts are pushed toward each other as the fixing projections penetrate into the bone parts, since the bone parts are displaced in the direction of the points of attachment of the fixing projections to the engaging elements and since the spacing of the fixing projections at the base points of the fixing projections is smaller than at the free ends.

### BRIEF DESCRIPTION OF THE DRAWINGS

The following description of exemplary embodiments of the invention serves for a more detailed explanation in conjunction with the drawing, in which:

FIG. 1 shows a perspective view of two schematically represented bone parts with an implant for their connection, before the tensioning of the engaging elements that can be applied to the bone parts;

FIG. 2 shows an enlarged view of the implant of FIG. 1 in the region of a bridge connecting two tensioning elements, in the direction of the arrow A in FIG. 1, and

FIG. 3 shows an enlarged view of a detail of the region B in FIG. 2.

### DETAILED DESCRIPTION

The ensuing detailed description provides exemplary embodiments only, and is not intended to limit the scope, applicability, or configuration of the invention. Rather, the ensuing detailed description of the exemplary embodiments will provide those skilled in the art with an enabling description for implementing an embodiment of the invention. It should be understood that various changes may be made in the function and arrangement of elements without departing from the spirit and scope of the invention as set forth in the appended claims.

The two bone parts 1, 2 to be connected to each other may be, for example, two plate-shaped parts of the cranial bone or the parts of the costal arches separated from each other in a heart operation, which are separated along the sternum. In the case of the exemplary embodiment represented, the bone parts 1, 2 are schematically represented in a very simplified form; all that is important is that the bone parts 1, 2 are to be fixed in relation to each other at the same height and brought together along an interspace 3 between the bone parts 1, 2.

This fixing is performed with the aid of an implant 4, which comprises a rectangular, first engaging plate 5 and a likewise rectangular, second engaging plate 6. The first engaging plate 5 has tooth-shaped fixing projections 7 that protrude perpendicularly from it, point in the direction of the second engaging plate 6 and are arranged along the edges of the first engaging plate 5, and in the same way fixing projections 8 are disposed on the second engaging plate 6, pointing in the direction of the first engaging plate 5. All the fixing projections 7, 8 are pointed and are therefore suitable for penetrating into the bone material when the engaging plates 5, 6 are pressed against the bone parts 1, 2. In this case, all the fixing projections 7, 8 are inclined such that the spacing of the fixing projections on opposite edges of the engaging plates becomes greater from the base points of the fixing projections 7, 8 on the engaging plates 5, 6 toward the free ends of the fixing projections 7, 8, that is to say the fixing projections 7, 8 are inclined slightly outward. This has the effect that, when the engaging plates 5, 6 are tensioned with respect to each other and the fixing projections 7, 8 penetrate into the bone parts 1, 2, the latter are brought together, that is to say are tensioned with respect to each other in the plane of the engaging plates 5, 6.

Projecting from the first engaging plate 5 are two tensioning pins 9, 10, which are spaced apart from each other and lie in the longitudinal center plane of the engaging plate 5, run parallel to each other and the length of which is much greater than the thickness of the bone parts 1, 2 to be connected to each other. These tensioning pins 9, 10 pass through openings 11, 12 in the second engaging plate 6; this second engaging plate 6 is mounted on the tensioning pins 9, 10 such that it can be displaced along them.

In the lower part, toward the first engaging plate 5, the tensioning pins 9, 10 are profiled, for example have ribs 13 running in the circumferential direction, and these serve for the fixing of the second engaging plate 6 in different positions along the tensioning pins 9, 10. For this purpose, the openings 11, 12 have radial incisions, by which the edge regions 14 adjoining the openings 11, 12 are subdivided into individual elastically bendable tongues, which elastically slide past the ribs 13 when the second engaging plate 6 is displaced in the direction of the first engaging plate 5, and remain in a position that makes pushing back impossible.

The portion of the tensioning pins 9, 10 that is away from the first engaging plate 5 does not have any such ribs 13, but is smooth, and the free ends of the two tensioning pins 9, 10 are pushed into bores 15, 16 of a bridge 17 that connects the two tensioning pins 9, 10 to each other and ends with the free ends of the tensioning pins 9, 10. The bridge 17 is deformed in the region of the bores 15, 16 by a crimping tool in such a way that the bridge 17 is held on the tensioning pins 9, 10 such that it cannot be displaced in the axial direction; this crimping produces crimping lines 18 on the outside of the bridge 17 in the form of peripheral grooves (FIG. 3).

The bridge 17 fixes the two tensioning pins 9, 10 in relation to each other at the same spacing as that at which the tensioning pins 9, 10 are held on the first engaging plate 5. On its side facing the two engaging plates 5, 6, the bridge 17 has an engaging surface 19, which is of an arcuate shape in cross-section and is disposed exactly between the two bores 15 and 16, so that the lowest point of the engaging surface 19 lies exactly midway between the bores 15 and 16.

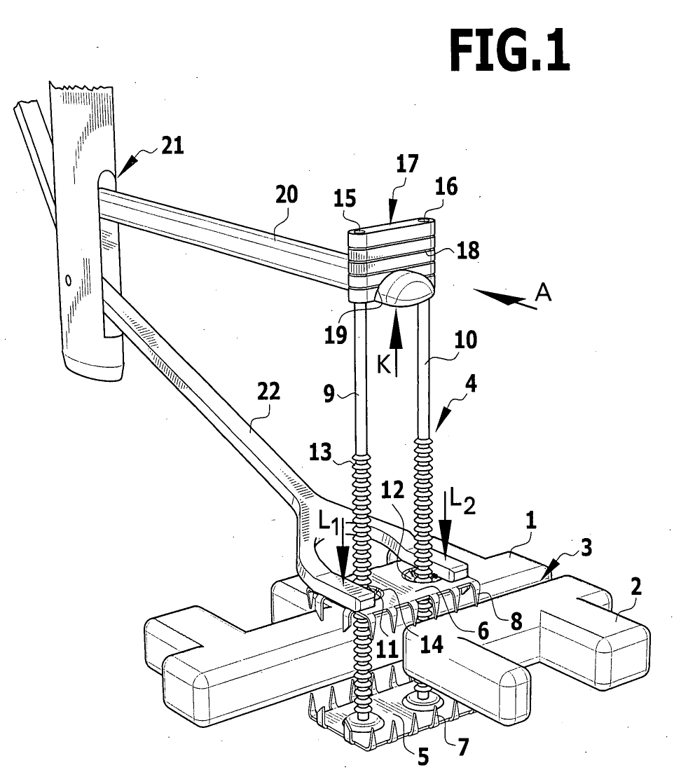

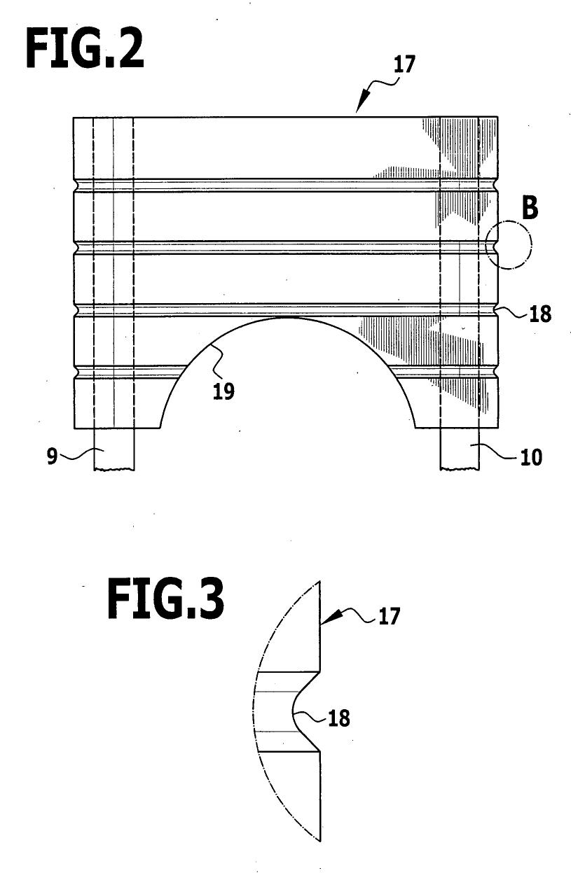

This engaging surface 19 forms a supporting surface for an upper tensioning tool 20 of a tensioning device 21, which in FIG. 1 is represented only very schematically and can be applied with a lower tensioning tool 22 to the upper side of the second engaging plate 6, to be precise right alongside the openings 11, 12 and outside the two tensioning pins 9, 10. In this way, the tensioning tools 20 and 22 can make engaging contact at three points altogether in the plane defined by the tensioning pins 9, 10, so that when the tensioning tools 20 and 22 are moved apart forces are introduced into the implant 4 at three points, to be specific an upwardly directed counterforce K midway between the two tensioning pins 9, 10 on the engaging surface 19 of the bridge 17 and tensioning forces L₁ and L₂, directed toward the first engaging plate, in the region between the narrow edges of the second engaging plate 6 and the openings 11, 12 on the upper side of the second engaging plate 6. This makes it possible to displace the second engaging plate 6 along the tensioning pins 9, 10 toward the first engaging plate 5 without tilting and, as a result, to tension the bone parts 1, 2 between the two engaging plates 5, 6.

Consequently, the bridge 17 not only assumes the task of stabilizing the tensioning pins 9, 10 with respect to each other and of covering the free ends of the tensioning pins 9, 10, but at the same time the bridge 17 acts as an engaging element for the tensioning tool 20 of the tensioning device 21.

After the tensioning of the two engaging plates 5, 6, parts of the tensioning pins 9, 10 protruding beyond the second engaging plate 6 may be removed, for example with the aid of a cutting device. It is in this case ensured by the bridge 17 that the removed parts remain as a one-piece unit, so that the risk of parts of the tensioning pins 9, 10 remaining in the operating area when they are cut off is reduced.

Furthermore, in this cutting operation the tensioning pins 9, 10 may be held on the bridge 17, optionally also with the aid of a suitable tool that acts directly on the bridge 17.

It should now be appreciated that the present invention provides advantageous methods and apparatus for the fixing together of two bone parts.

Although the invention has been described in connection with various illustrated embodiments, numerous modifications and adaptations may be made thereto without departing from the spirit and scope of the invention as set forth in the claims.

What is claimed is:

1. Implant for fixing two bone parts to be connected to each other, comprising:
a first plate-shaped engaging element for engaging inner surfaces of the bone parts;
at least two tensioning elements fixed on said first engaging element and projecting transversely from it;
a second plate-shaped engaging element for engaging outer surfaces of the bone parts, which can be tensioned with respect to the first engaging element by means of the at least two tensioning elements extending through an interspace between the bone parts and passing through corresponding openings in the second engaging element; and
a bridge for connecting the at least two tensioning elements to each other at ends of the at least two tensioning elements opposite from the first engaging element.

2. Implant in accordance with claim 1, wherein the bridge is connected to the tensioning elements so as to be non-displaceable in a tensioning direction.

3. Implant in accordance with claim 1, wherein the tensioning elements are pins.

4. Implant in accordance with claim 2, wherein the tensioning elements are pins.

5. Implant in accordance with claim 2, wherein the bridge is connected to the tensioning elements in a non-displaceable manner by crimping.

6. Implant in accordance with claim 5, wherein the tensioning elements are inserted in bores in the bridge.

7. Implant in accordance with claim 1, wherein:
the bridge has an engaging surface for a tensioning device having two tensioning tools, one of said tensioning tools is adapted for engagement with the engaging surface and the other of said tensioning tools is adapted for engagement with an outer surface of the second engaging element; and
the tensioning device tensions the second engaging element with respect to the first engaging element when the tensioning tools are pushed apart.

8. Implant in accordance with claim 7, wherein:
the at least two tensioning elements comprise two tensioning elements; and
the engaging surface and the tensioning tool adapted for engaging said engaging surface are shaped in such a way that the tensioning tool is supported on the bridge midway between the two tensioning elements.

9. Implant in accordance with claim 8, wherein:
the tensioning tool adapted for engaging the second engaging element is adapted to be applied at two points on the second engaging element; and
the two points are disposed in a plane defined by said at least two tensioning elements at a distance from each other and away from a midpoint between the two tensioning elements.

10. Implant in accordance with claim 8, wherein the engaging surface of the bridge extends in an arcuate form.

11. Implant in accordance with claim 1, wherein:
each of said engaging elements carry fixing projections protruding therefrom and respectively pointing toward the other engaging element; and
said fixing projections are inclined in such a way that their spacing from corresponding fixing projections on an opposite side of the engaging element increases from the engaging element toward a free end of the fixing projections.

12. Implant in accordance with claim 1, wherein portions of said tensioning elements protruding beyond an outer surface of said second engaging element are cut off subsequent to said tensioning.

13. Implant in accordance with claim 12, wherein the portions of the tensioning elements protruding beyond an outer surface of said second engaging element and said bridge are cut off as a unit.

14. Implant in accordance with claim 12, wherein the portions of the tensioning elements protruding beyond an outer surface of said second engaging element are held on the bridge when said portions of the tensioning elements are cut off.

15. A method for fixing two bone parts to be connected to each other, comprising:
positioning a first plate-shaped engaging element against inner surfaces of the bone parts, with at least two tensioning elements fixed on said first engaging element and extending through an interspace between the bone parts;
positioning a second plate-shaped engaging element against outer surfaces of the bone parts with said at least two tensioning elements passing through openings in said second engaging element;
connecting the at least two tensioning elements to each other at ends of the at least two tensioning elements opposite from the first engaging element using a bridge; and
tensioning the second engaging element with respect to the first engaging element.

16. A method in accordance with claim 15, wherein the bridge is connected to the tensioning elements so as to be non-displaceable in a tensioning direction.

17. A method in accordance with claim 15, wherein the tensioning elements are pins.

18. A method in accordance with claim 16, wherein the tensioning elements are pins.

19. A method in accordance with claim 15, wherein said connecting comprises inserting the ends of the tensioning elements into bores in the bridge.

20. A method in accordance with claim 16, further comprising:
crimping the bridge to the ends of tensioning elements.

21. A method in accordance with claim 15, further comprising:
employing a tensioning de-vice having two tensioning tools for tensioning the second engaging element with respect to the first engaging element; wherein:
the bridge has an engaging surface;
one of said tensioning tools is adapted for engagement with the engaging surface and the other of said tensioning tools is adapted for engagement with an outer surface of the second engaging element; and
the tensioning device tensions the second engaging element with respect to the first engaging element when the tensioning tools are pushed apart.

22. A method in accordance with claim 21, wherein:
said at least two tensioning elements comprise two tensioning elements; and
the engaging surface and the tensioning tool adapted for engaging said engaging surface are shaped in such a way that the tensioning tool is supported on the bridge midway between the two tensioning elements.

23. A method in accordance with claim 22, wherein:
the tensioning tool adapted for engaging the second engaging element is adapted to be applied at two points on the second engaging element; and
the two points are disposed in a plane defined by said at least two tensioning elements at a distance from each other and away from a midpoint between the two tensioning elements.

24. A method in accordance with claim 22, wherein the engaging surface of the bridge extends in an arcuate form.

25. A method in accordance with claim 15, wherein:
each of said engaging elements carry fixing projections protruding therefrom and respectively pointing toward the other engaging element; and
said fixing projections are inclined in such a way that their spacing from corresponding fixing projections on an opposite side of the engaging element increases from the engaging element toward a free end of the fixing projections.

26. A method in accordance with claim 15, further comprising:
cutting off portions of said tensioning elements protruding beyond an outer surface of said second engaging element subsequent to said tensioning.

27. A method in accordance with claim 26, wherein the portions of the tensioning elements protruding beyond an outer surface of said second engaging element and said bridge are cut off as a unit.

28. A method in accordance with claim 26, further comprising:
holding the portions of the tensioning elements protruding beyond an outer surface of said second engaging element on the bridge during said cutting.
